(19) United States
(12) Patent Application Publication
(10) Pub. No.: US 2013/0053600 A1
(43) Pub. Date: Feb. 28, 2013

(54) METHOD FOR PREPARING A CARBOXYLIC ACID ESTER

(71) Applicants: Carlos Eduardo Marenco, Campinas (BR); Wilson Martins, Campinas (BR); Alexandre Tresmondi, Campinas (BR); Joël Schwartz, Caluire (FR)

(72) Inventors: Carlos Eduardo Marenco, Campinas (BR); Wilson Martins, Campinas (BR); Alexandre Tresmondi, Campinas (BR); Joël Schwartz, Caluire (FR)

(73) Assignee: RHODIA POLIAMIDA E ESPECIALIDADES LTDA, São Paulo (BR)

(21) Appl. No.: 13/576,915

(22) PCT Filed: Feb. 3, 2011
(86) PCT No.: PCT/IB2011/000172

§ 371 Date: Oct. 11, 2012

(30) Foreign Application Priority Data
Feb. 4, 2010 (FR) 1050768

(51) Int. Cl. C07C 67/08; C09K 3/00; B01D 3/32

(52) U.S. Cl. 560/265; 422/187; 252/182.12; 252/183.11

(57) ABSTRACT

A method of manufacturing a carboxylic acid ester is described. Also described, is a method for manufacturing a carboxylic acid ester by reacting a carboxylic acid and an alcohol, wherein the reaction is performed in the presence of an excess of carboxylic acid.

The present invention relates to a process for manufacturing a carboxylic acid ester.

The invention especially targets the preparation of acetic acid esters and more particularly ethyl acetate.

Acetic acid esters, in particular ethyl acetate, are generally used as organic solvents. In particular, ethyl acetate is especially used in the cosmetics and fragrance fields, and in adhesives, paints and varnishes.

Depending on the application targeted, a higher or lower purity is required and it is common to request that the amount of acetic acid present in the ethyl acetate be less than 0.01% by weight.

Thus, the processes for manufacturing ethyl acetate must result in a quality product. Given that ethyl acetate is a standard, high-volume consumer product, it is important that its manufacturing process be as effective as possible, in terms of productivity and energy balance.

A process for preparing ethyl acetate is described, in U.S. Pat. No. 4,481,146, in which the molar ratio between the acetic acid and the alcohol is between 1 and 5.

However, the productivity of this process and also the reduction in the energy costs for production can be improved.

The objective of the present invention is to provide an improved process for preparing a carboxylic acid ester, in terms of process economics.

Therefore, one subject of the present invention is a process for manufacturing a carboxylic acid ester by reaction between a carboxylic acid and an alcohol, especially in the presence of an acid catalyst, in which the reaction is carried out so that the carboxylic acid/alcohol molar ratio is at least equal to 7.

In accordance with the process of the invention, an esterification reaction of the carboxylic acid, preferably of acetic acid, is carried out by an alcohol preferably in the presence of an acid catalyst. It has been found, according to the invention, that when the carboxylic acid/alcohol molar ratio is very high, that is to say at least equal to 7, the process is improved as regards the energy costs for operation.

Moreover, the requirements of maximum content of carboxylic acid in the carboxylic acid ester obtained are respected.

A carboxylic acid, an alcohol and preferably an acid catalyst take part in the process of the invention.

Although the process of the invention is perfectly suited to acetic acid, it may also be suitable for aliphatic carboxylic acids, having from 1 to 6 carbon atoms.

The carboxylic acid is advantageously introduced pure or in highly concentrated aqueous solution. The process of the invention does not exclude the presence of water in the carboxylic acid. However, it is preferable to use pure carboxylic acid due to the subsequent need to remove the water present in the carboxylic acid ester obtained at the end of the process.

The alcohol is preferably an alcohol with a linear or branched alkyl chain having from 1 to 6 carbon atoms or an alcohol with a cycloalkyl chain having 5 or 6 carbon atoms.

In particular alcohols with a low boiling point, especially below 170° C., preferably below 165° C., are preferred.

Thus, the alcohol with an alkyl chain is advantageously chosen from ethanol, butanol and n-propanol, preferably ethanol.

The alcohol with a cycloalkyl chain is preferably cyclohexanol. According to the invention, the molar ratio between the carboxylic acid and the alcohol is at least equal to 7, preferably at least equal to 9, and more preferably still at least equal to 16.

The upper limit, for economic reasons, is advantageously chosen to be less than 25, preferably less than 20.

Thus, the molar ratio between the carboxylic acid and the alcohol is preferably between 9 and 25, especially between 16 and 25, and more preferably between 9 and 20, especially between 16 and 20. The precisely defined ratio corresponds to the molar ratio of the reactants at the start of the reaction.

The catalyst that takes part in the process of the invention is preferably a protonic acid.

According to a first embodiment, the catalyst is a heterogeneous acid catalyst. The heterogeneous acid catalysts of the invention are preferably sulfonic resins or zeolites. The zeolites that may be used are, for example, those mentioned in Application WO 2007/099071. The resins that are suitable for the present invention may have a polystyrene or polyacrylic backbone that bears sulfonic functional groups. Thus, use may be made of commercial sulfonic resins, resins sold under various trade names. Mention may be made, inter alia, of the following esterification resins: Amberlyst® 15 from Rohm and Haas, Amberlite® IR-120 H from Rohm and Haas, Lewatit® 2631 and K1431 from Bayer. The acidity of these resins is, for example, between 1 and 10 eq/kg (H+). These resins are especially used in a fixed or fluidized bed, preferably in a fixed bed.

According to a second embodiment, the catalyst is a homogeneous strong acid catalyst. This second embodiment is a preferred embodiment of the invention.

The expression “strong acid” is understood to mean, within the present invention, an acid having a pKₐ in water of less than 2, preferably of less than 1.

The pKₐ is defined as follows: pKₐ=−log Kₐ, Kₐ being the ionic dissociation constant of the acid/base pair at ambient temperature (generally 25° C.), when water is used as the solvent.

Among the acids that correspond to this definition, it is preferable to use an acid that does not result in parasitic reactions that hinder the esterification process, and in particular that do not have an oxidizing nature, such as nitric acid.

As a homogeneous strong acid, mention may more particularly be made of sulfuric acid, sulfonic acids and mixtures thereof.

As sulfonic acids, mention may especially be made of fluorosulfonic acid, chlorosulfonic acid or trifluoromethanesulfonic acid, methanesulfonic acid, ethanesulfonic acid, camphenesulfonic acid, benzenesulfonic acid, toluenesulfonic acids, xylenesulfonic acids and naphthalenesulfonic acids.

Among these acids, the preferred catalyst is chosen from para-toluenesulfonic acid or methanesulfonic acid, preferably methanesulfonic acid.

The amount of catalyst introduced is such that the amount present in the reactor is preferably between 0.1 and 2% by weight relative to the reaction medium.

It is possible to introduce other compounds into the reaction, for example corrosion inhibitors. These may especially be copper (II) sulfate.

In accordance with the process of the invention, the esterification reaction may be carried out in continuous mode or in batch mode.

According to one preferred embodiment of the invention, the process is a continuous process.

According to one preferred embodiment of the invention, the following steps are used:
a. the carboxylic acid and the alcohol are reacted, in the presence of an acid catalyst, at a temperature at least equal to 50° C.;
b. the reaction mixture obtained previously is then subjected to a distillation operation in order to obtain:
   a vapor stream comprising predominantly the carboxylic acid ester, at the distillation tops; and
   a medium comprising predominantly carboxylic acid, at the distillation bottom; and
c. the medium at the distillation bottom is withdrawn and reintroduced at the start of reaction a.

According to another still more preferred embodiment of the invention, the following steps are used:
a. the carboxylic acid and the alcohol are reacted, in the presence of an acid catalyst, at a temperature at least equal to 50° C., and at a pressure such that the reaction mixture is in the liquid state;
b. the liquid reaction mixture obtained previously is then subjected to a distillation operation in order to obtain:
   a vapor stream comprising predominantly the carboxylic acid ester, at the distillation top; and
   a medium comprising predominantly carboxylic acid, at the distillation bottom; and
c. the medium at the distillation bottom is withdrawn and reintroduced at the start of reaction a.

In the process of the invention, the carboxylic acid and the alcohol may be introduced alone or as a mixture. Preferably, the carboxylic acid and the alcohol are introduced as a mixture.

Advantageously, the reaction temperature is between 50 and 150° C., preferably between 100 and 130° C.

The reaction is preferably carried out at atmospheric pressure. A pressure slightly above or below atmospheric pressure may also be suitable. The reaction is advantageously carried out at a pressure such that the reaction mixture is in the liquid state. Thus, the process of the invention may be implemented, for example, at an absolute pressure between 0.5 and 5 bar absolute, and more preferably still between 1.5 and 5 bar.

Advantageously, the reaction mixture obtained at the end of the reaction is subjected to a distillation operation. This distillation operation is preferably carried out in a distillation column. The feed point where the reaction mixture is introduced is, in general, substantially at the mid-height of the distillation column. It may also be located lower down, at a height between the mid-height of the column and the bottom of the column.

The temperature at the distillation bottom is preferably between 50 and 150° C., preferably between 100 and 130° C.

The defined pressure at the distillation top is preferably between 0.5 and 5 bar absolute, advantageously the pressure at the distillation top is between 1 and 2 bar absolute.

This distillation operation makes it possible to obtain a vapor stream comprising predominantly the carboxylic acid ester at the distillation top and a medium at the distillation bottom comprising predominantly the carboxylic acid.

The term “predominantly” is understood to mean that the medium consists of at least 75% by weight, preferably at least 85% by weight, of the compound in question.

In the process of the invention, the vapor stream at the distillation top especially comprises:
75 to 98% by weight of carboxylic acid ester;
0 to 17% by weight of water;
0 to 8% by weight of alcohol; and
traces of carboxylic acid.

The vapor stream at the distillation top preferably comprises:
85 to 95% by weight of carboxylic acid ester;
0 to 10% by weight of water;
0 to 5% by weight of alcohol; and
traces of carboxylic acid.

The expression “traces of carboxylic acid” is understood to mean less than 0.02% by weight, preferably between 0.001 and 0.02% by weight, of carboxylic acid.

In the process of the invention, the medium at the distillation bottom especially comprises:
75 to 98% by weight of carboxylic acid;
0 to 15% by weight of carboxylic acid ester;
0 to 10% by weight of water; and
0 to 3% by weight of alcohol.

The medium at the distillation bottom preferably comprises:
80 to 95% by weight of carboxylic acid;
2 to 15% by weight of carboxylic acid ester;
2 to 8% by weight of water; and
0.1 to 2% by weight of alcohol.

The medium at the distillation bottom may, moreover, contain up to 2% by weight of catalyst.

According to this particular embodiment of the invention, the medium at the distillation bottom is withdrawn and recycled by reintroduction upstream of or during the reaction, preferably upstream of the reaction.

The term “upstream” is understood to mean that the medium at the distillation bottom is reintroduced into the mixture of carboxylic acid and alcohol before it has reacted.

The ratio between the flow rate of the recycle stream and the feed flow rate of the reaction mixture (carboxylic acid+alcohol) is advantageously between 4 and 20, preferably between 5 and 15.

In this embodiment, the carboxylic acid/alcohol molar ratio is either the ratio in the mixture comprising the carboxylic acid and the alcohol before the reaction when the reactants are introduced as a mixture, or the ratio at the start of the reaction when the reactants are introduced separately into the reaction. This ratio takes into account the supply of carboxylic acid and of alcohol originating from the recycling.

Advantageously, the carboxylic acid/alcohol molar ratio at the end of the reaction is between 40 and 120, preferably between 60 and 100.

The reaction mixture, which is preferably liquid, resulting from the esterification reaction is distilled and makes it possible to obtain a vapor stream at the top of the column. The vapor stream is cooled and converted to liquid form by lowering its temperature to a temperature, for example between 15° C. and 40° C., by passing it through one or more condensers.

The liquid stream thus obtained may then be sent to a liquid phase separation means, preferably a settling tank that separates the organic phase containing predominantly the carboxylic acid ester and the aqueous phase containing predominantly water.

The organic phase is, advantageously, partly reintroduced at the distillation top in order to ensure that the column is operating under reflux. The reflux ratio is preferably between 1 and 8, advantageously between 2 and 6. The remainder of the organic phase constitutes the expected carboxylic acid ester and may be retreated, especially by distillation, to remove the residual traces of water and alcohol.

The aqueous phase resulting from the separation is treated, for example by distillation, so that a phase comprising predominantly alcohol and a phase comprising predominantly water are recovered. The phase comprising predominantly alcohol may advantageously be recycled by reintroduction at the start of the reaction.

According to another more specific aspect, another subject of the present invention is a device for implementing the process of the invention.

This device, which most often takes the form of an installation of industrial dimensions, comprises:
a reactor; and
a distillation column for which:
   the top part is connected to means for treating the vapor stream originating from the top of the distillation column, namely at least one condenser, the outlet of which is connected to a liquid/liquid phase separator, preferably a settling tank; and
   the bottom part is connected to means for withdrawing and recycling the liquid stream originating from the bottom of the distillation column to the reactor.

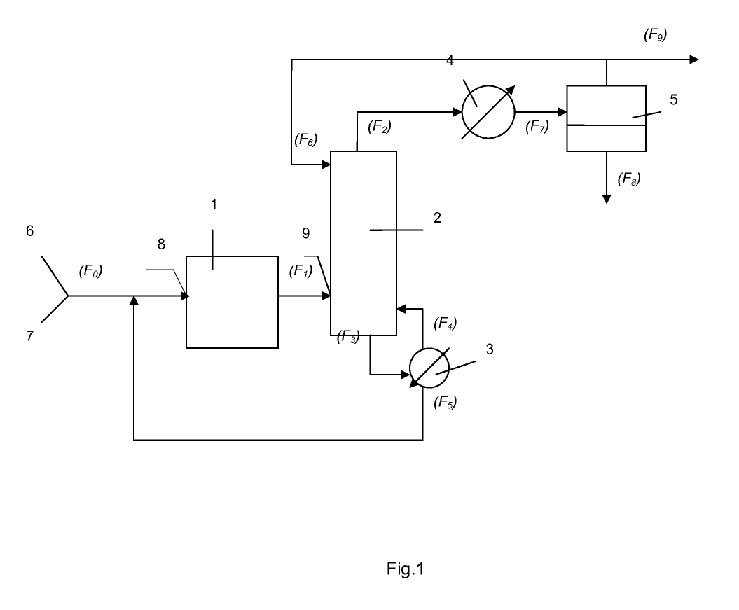

The invention will be explained in further detail by means of the following description, given in reference to FIG. 1.

FIG. 1 is a schematic representation of a preferred device for the implementation of the process according to the invention comprising a reactor 1, a distillation column 2 the top part of which is connected to means for treating the vapor stream originating from the distillation column 2, namely at least one condenser 4 the outlet of which is connected to a settling tank 5.

The alcohol 6 and the carboxylic acid 7 form a preferably liquid stream (F₀) that is introduced at 8 into a reactor 1.

The reactor is preferably adiabatic. It may be of the perfectly stirred type or of the plug flow type, preferably of the plug flow type.

The preferably liquid stream (F₁) resulting from the reaction is introduced at 9 into a distillation column 2.

This step aims to obtain, at the bottom, a liquid stream (F₃) comprising predominantly the carboxylic acid and, at the top, a vapor stream (F₂) comprising predominantly the expected carboxylic acid ester.

A person skilled in the art is perfectly capable of choosing the means to be used depending on the separation to be carried out.

Only the following will be mentioned. The size (especially the diameter) of the distillation columns depends on the stream circulating and on the internal pressure.

They are therefore sized mainly according to the flow rate of mixture to be treated. The internal parameter, which is the number of theoretical plates, is especially determined by the purity of the starting compound and the purities of the products that have to be obtained at the distillation top and at the distillation bottom.

It will be specified that the column may be packed either with plates or with structured or woven packing, as is perfectly well known to a person skilled in the art.

Once the installation is determined, a person skilled in the art adjusts the operating parameters of the column.

Thus, the distillation column could advantageously, but non-limitingly, be a column having the following specifications:
number of theoretical plates: from 1 to 40, preferably from 2 to 20; and
reflux ratio R between 1 and 8, preferably between 2 and 6.

The reflux ratio is defined as the ratio of the flow rate of material reinjected from the top of the column to the inside of the column to the flow rate of organic phase actually exiting the settling tank.

In order to carry out the distillation, the supply of heat at the bottom of the column may be especially made by a shell and tube heat exchanger, a plate heat exchanger, a coil heat exchanger or by any other equivalent device. The heating may be carried out using steam or a heat transfer fluid.

One preferred embodiment consists in heating the mixture at the distillation bottom in a heat exchanger 3 by removing a stream (F₄) at the bottom which flows in a loop. More precisely, the stream (F₃) exits at the distillation bottom and a fraction (F₄) crosses a heat exchanger from the bottom to the top and on exiting the exchanger is introduced in the form of a liquid/vapor mixture into the lower part of the distillation column.

Another embodiment consists in carrying out a forced circulation of the stream (F₃) in the exchanger using a pump.

The stream (F₅) exiting the exchanger is retransported upstream for example by means of pumps. The ratio of the flow rate of the stream (F₅) to the flow rate of the stream (F₀) is preferably between 4 and 20.

The vapor stream (F₂) at the top of the column, comprising predominantly the carboxylic acid ester, is condensed so as to recover a liquid stream one fraction (F₆) of which is introduced, sideways, at the top of the column, to ensure the reflux in the column, and the other fraction (F₉) may be treated in a subsequent purification step of the carboxylic acid ester.

At the distillation top, the stream comprising mainly the carboxylic acid ester is recovered from the stream (F₂) by condensation, for example by passing through one or more condensers 4.

The vapor phase (F₂) is cooled and converted to liquid form via cooling by lowering its temperature to a temperature for example between 15° C. and 40° C.

This operation is carried out by passing through a condenser, which is a conventional device, for example a tubular heat exchanger fed with a heat transfer fluid (generally water) maintained at a temperature close to the chosen cooling temperature.

The number and size of the condensers are chosen as a function of the cooling capacities of the coolants circulating in the condensers.

In the case of condensers in series, the vapor phase exiting the first condenser is introduced into the second condenser.

The liquid stream (F₇) is recovered at the outlet of the condenser(s).

The liquid stream (F₇) is introduced into a settling tank 5 that separates the aqueous phase (F₈) from the organic phase (F₉).

The phase (F₈) may be retreated by passing into an entrainment device (not shown) that makes it possible to recover, on the one hand, a phase comprising predominantly water and, on the other hand, a phase comprising predominantly alcohol and carboxylic acid ester as a mixture.

The expected ester is present in the stream (F₉), and may optionally be purified, especially by distillation, in order to remove the residual amounts of alcohol and water.

The process of the invention is particularly beneficial due to the advantages that it provides.

One of the advantages of the present invention is that almost all of the alcohol is converted to carboxylic acid ester.

Thus, a smaller amount of alcohol is present in the vapor phase (F₂) at the top of the column comprising predominantly the carboxylic acid ester, preferably the acetic acid ester. Consequently, less energy is consumed for the separation of the alcohol and the carboxylic acid ester.

The following examples illustrate the invention without however limiting it.

In the examples, the degree of conversion (DC) is defined, which corresponds to the ratio between the number of moles of substrate converted and the number of moles of substrate used.

### EXAMPLES

For a better understanding, the examples below are described in reference to FIG. 1. The following examples are given for a final production of 100 kg/h of ethyl acetate.

### Example 1 (Ex. 1)

An esterification reactor 1 is fed by simultaneous introduction of a liquid stream of ethanol 6 with a flow rate of 56 kg/h and a liquid stream of acetic acid 7 with a flow rate of 69 kg/h. The total flow rate of the liquid feed stream is called flow rate (F₀).

Also introduced into the esterification reactor 1 are 2.3 kg of methanesulfonic acid as catalyst. The esterification reactor 1 has a capacity of 230 kg.

Methanesulfonic acid is regularly injected and purged so as to maintain the catalytic activity.

The inlet temperature of the reactor is 117° C. and the pressure in the reactor is regulated so that the reaction mixture in the reactor is liquid, i.e. a pressure at the top of the reactor of 2 bar absolute. The reactor is adiabatic.

The liquid stream (F₁) exiting the esterification reactor then feeds an esterification column 2. This column comprises 16 theoretical plates. It operates at a pressure of 1.5 bar absolute and at a temperature at the bottom of the column of 118° C.

The stream (F₅) originating from the bottom of the esterification column and containing predominantly acetic acid is recycled to the inlet of the esterification reactor 1.

The recycling of said stream (F₅) is carried out so that the molar ratio of acetic acid to ethanol is 16. Under these conditions, the weight ratio between the recycling flow rate (F₅) and the feed flow rate (F₀) is 12.

The stream (F₂) at the top of the column containing predominantly ethyl acetate, the water of reaction and 2.0% by weight of unconverted ethanol is condensed in a heat exchanger 4 then sent to a settling tank 5 in which two phases are separated.

The aqueous phase (F₈) contains predominantly water and, in a minor amount, ethanol and ethyl acetate. This aqueous phase (F₈) is sent to a distillation column with a view to separating the water and a stream comprising predominantly the unconverted ethanol and ethyl acetate, which is recycled to the esterification reactor.

The organic phase (F₆+F₉) exiting the settling tank 5 contains predominantly ethyl acetate and, to saturation, water and ethanol.

A portion (F₆) of this organic phase is sent back to the top of the column to ensure the reflux with an (F₆)/(F₉) reflux ratio of 3.6.

The other portion (F₉) of the organic phase is then distilled so as to remove the water and the ethanol that it contains in order to obtain the desired quality of the final product. The ethanol content in the ethyl acetate after this final distillation is less than 0.03% by weight.

### Example 2 (Ex. 2)

The above process described in Example 1 is reproduced for an acetic acid/alcohol molar ratio of 7.

### Example 3 (Ex. 3)

The above process described in Example 1 is reproduced for an acetic acid/alcohol molar ratio of 25.

### Example 4 (Ex. 4)

The above process described in Example 1 is reproduced for an acetic acid/alcohol molar ratio of 10.

### Example 5 (Ex. 5)

The above process described in Example 1 is reproduced for an acetic acid/alcohol molar ratio of 12.

### Comparative Example (Comp. Ex.)

The above process described in Example 1 is reproduced for an acetic acid/alcohol molar ratio of 5.

### Results:

The results are presented in the table below, in which the comparison between the examples is understood to mean with all the other parameters moreover being equal. The overall steam consumption of the whole of the installation was measured for each of the examples.

| | | Comp. | | | | | |
|---|---|---|---|---|---|---|---|
| Parameters | Units | ex. | Ex. 2 | Ex. 4 | Ex. 5 | Ex. 1 | Ex. 3 |
| Acetic acid/ | mol/mol | 5 | 7 | 10 | 12 | 16 | 25 |
| ethanol ratio | | | | | | | |
| (F₅)/(F₀) | kg/kg | 2.8 | 4.4 | 6.7 | 8.2 | 12 | 20 |
| DC ethanol | % | 87.5 | 86.9 | 86.5 | 85.8 | 83.7 | 76.1 |
| Ethanol at top of | wt % | 3.4 | 2.9 | 2.4 | 2.2 | 2.0 | 1.8 |
| column (F₂) | | | | | | | |
| Ethanol after | wt % | 0.6 | 0.03 | 0.03 | 0.03 | 0.03 | 0.03 |
| final distillation | | | | | | | |
| Overall steam | MWh/t | 2.5 | 2.3 | 1.6 | 1.6 | 1.5 | 1.5 |
| consumption | | | | | | | |

The increase in the acetic acid/ethanol molar ratio at the inlet of the esterification reactor makes it possible to decrease the energy consumption of the final ethyl acetate distillation column and therefore the overall consumption of the unit. Moreover, it is observed that the amount of ethanol in the ethyl acetate after the final distillation is substantially higher for the comparative example (twenty times higher). The increase in the acetic acid/ethanol molar ratio at the inlet of the esterification reactor therefore also makes it possible to improve the final quality of the ethyl acetate.

1. A process for manufacturing a carboxylic acid ester, the process comprising conducting a reaction between a carboxylic acid and an alcohol, wherein the reaction is carried out so that the carboxylic acid/alcohol molar ratio is at least equal to 7.

2. The process as defined by claim 1, wherein the carboxylic acid is an aliphatic carboxylic acid having from 1 to 6 carbon atoms.

3. The process as defined by claim 2, wherein the carboxylic acid is an acetic acid.

4. The process as defined by claim 1, wherein the alcohol is selected from the group consisting of an ethanol, a butanol, a n-propanol and a cyclohexanol.

5. The process as defined by claim 4, wherein the alcohol is ethanol.

6. The process as defined by claim 1, wherein the carboxylic acid/alcohol molar ratio is from 16 to 25.

7. The process as defined by claim 1, further comprising the following steps:
a. reacting the carboxylic acid and the alcohol, in the presence of an acid catalyst, at a temperature at least equal to 50° C., and at a pressure such that a resulting reaction mixture is in a liquid state;
b. subjecting the liquid reaction mixture obtained previously to a distillation operation in order to obtain:
a vapor stream comprising predominantly the carboxylic acid ester, at the distillation top; and
a medium comprising predominantly the carboxylic acid, at the distillation bottom; and
c. withdrawing the medium at the distillation bottom and reintroducing it at the start of reaction a.

8. The process as defined by claim 7, wherein the process is continuous.

9. The process as defined by claim 8, wherein the medium at the distillation bottom is reintroduced at the start of the reaction so that the ratio between the flow rate of the recycle stream and the feed flow rate of the reaction mixture (carboxylic acid+alcohol) is from 4 to 20.

10. The process as defined by claim 7, wherein the reaction temperature is from 50° C. to 150° C.

11. The process as defined by claim 10, wherein the reaction is carried out at an absolute pressure from 0.5 bar absolute to 5 bar absolute.

12. The process as defined by claim 7, wherein the temperature at the distillation bottom is from 50° C. to 150° C.

13. The process as defined by claim 12, wherein a defined pressure at the distillation top is from 0.5 bar absolute to 5 bar absolute.

14. The process as defined by claim 7, wherein the catalyst is a strong protonic acid.

15. The process as defined by claim 14, wherein the catalyst is a homogeneous catalyst selected from the group consisting of a sulfuric acid, a sulfonic acid and a mixture thereof.

16. The process as defined by claim 14, wherein the catalyst is a heterogeneous catalyst that is a sulfonic resin.

17. The process as defined by claim 7, wherein the vapor stream at the distillation top comprises:
75% to 98% by weight of carboxylic acid ester;
0% to 17% by weight of water;
0% to 8% by weight of alcohol; and
traces of carboxylic acid.

18. The process as defined by claim 7, wherein the medium at the distillation bottom comprises:
75% to 98% by weight of carboxylic acid;
0% to 15% by weight of carboxylic acid ester;
0% to 10% by weight of water; and
0% to 3% by weight of alcohol.

19. A device for implementing the process as defined by claim 1, the device comprising:
a reactor; and
a distillation column for which:
a top part is connected to means for treating a vapor stream originating from the top of the distillation column, an outlet of which is connected to a liquid/liquid phase separator, and
bottom part is connected to means for withdrawing and recycling a liquid stream originating from the bottom of the distillation column to the reactor.

20. A medium at a distillation bottom of an operation for distilling a carboxylic acid ester obtained by reaction between a carboxylic acid and an alcohol, said medium comprising:
75% to 98% by weight, of carboxylic acid;
0% to 15% by weight, of carboxylic acid ester;
0% to 10% by weight, of water; and 0% to 3% by weight of alcohol.

21. The process as defined by claim 6, wherein the carboxylic acid/alcohol molar ratio is from 16 to 20.

22. The process as defined by claim 8, wherein the ratio between the flow of the recycle stream and the feed flow rate of the reaction mixture is from 5 to 15.

23. The process as defined by claim 10, wherein the reaction temperature is from 100° C. to 130° C.

24. The process as defined by claim 12, wherein the temperature at the distillation bottom is from 100° C. to 130° C.

25. The process as defined by claim 13, wherein the defined pressure at the distillation top is from 1 bar absolute to 2 bar absolute.

26. The process as defined by claim 15, wherein the homogeneous strong acid is para-toluenesulfonic acid or methane sulfonic acid.

27. The process as defined by claim 17, wherein the distillation top comprises from 85 to 95% by weight of carboxylic acid ester.

28. The process as defined by claim 17, wherein the distillation top comprises from 0% to 10% by weight of water.

29. The process as defined by claim 17, wherein the distillation top comprises from 0% to 5% by weight of alcohol.

30. The process as defined by claim 18, wherein the distillation bottom comprises from 80% to 95% by weight of carboxylic acid.

31. The process as defined by claim 18, wherein the distillation bottom comprises from 2% to 15% by weight of carboxylic acid ester.

32. The process as defined by claim 18, wherein the distillation bottom comprises from 2% to 8% by weight of water.

33. The process as defined y claim 18, wherein the distillation bottom from 0.1% to 2% by weight of alcohol.

34. The device as defined by claim 19, wherein the means for treating the vapor stream originating from the top of the distillation column comprises at least one condenser.

35. The device as defined by claim 19, wherein the liquid/liquid phase separator comprises a settling tank.

36. The medium as defined by claim 20, wherein the medium comprises from 80% to 95% by weight of carboxylic acid.

37. The medium as defined by claim 20, wherein the medium comprises from 2% to 15% by weight of carboxylic acid ester.

38. The medium as defined by claim 20, wherein the medium comprises from 2% to 8% by weight of water and from 0.1% to 2% by weight of alcohol.
